(19) United States
(12) Patent Application Publication
(10) Pub. No.: US 2006/0245541 A1
(43) Pub. Date: Nov. 2, 2006

(54) Mammography procedure and apparatus for reducing pain when compressing a breast

(76) Inventor: Leo J. Aubel, Lincolnshire, IL (US)

Correspondence Address: Leo J. Aubel, 111 Rivershire Lane, Lincolnshire, IL, 60069, US

(21) Appl. No.: 11/476,176

(22) Filed: Jun. 27, 2006

Related U.S. Application Data
(63) Continuation-in-part of application No. 11/145,400, filed on Jun. 3, 2005.
Application No. 11/145,400 is a continuation-in-part of application No. 10/789,001, filed on Feb. 26, 2004, now abandoned.

(51) Int. Cl. A61B 6/04

(52) U.S. Cl. 378/37

(57) ABSTRACT

A method and apparatus for compressing a patient's breast when using an X-ray mammography machine to take an image wherein said machine has a compression paddle and a bucky. A movable interface plate controllable by linear actuators is mounted on the bucky as an interface between the bucky and a patient's breast. The method includes steps wherein the compression paddle is moved downwardly to provide compression forces on the breast; the movement of the compression paddle is stopped at a position where less than the full desired compression of the breast is attained. Next, the movable interface plate is elevated under control of the linear actuators, upwardly against the breast to obtain the full desired compression. The upward movement of the interface plate functions to distribute and balance the compression and shear forces applied to the breast.

This application claims the priority filing date of U.S. provisional application Ser. No. 60/534,603 filed on Jan. 06, 2004 by the applicant herein. This application is a continuation-in-part of Ser. No. 10/789,001 filed on Feb. 26, 2004, and of Ser. No. 11/145,400 filed on Dec. 01, 2005.

### BACKGROUND OF INVENTION

This invention refers to a new method and apparatus used when compressing a patient's breast for taking a radiographic image. In mammography, compression of the breast is essential for good quality X-ray imaging. Firm compression spreads out the breast tissue, thereby reducing superimposed structures. Likewise, the low dosage X-rays used in mammography can more easily penetrate the thinner mass that results from compression and spreading of the breast.

Women are advised to undergo periodic mammography screening (examination) in an attempt to detect cancer at its earliest stages. Because of the harsh breast compression techniques, many patients consider mammograms to be uncomfortable, even painful, and studies show that patients are reluctant to schedule screenings after their first experience with the procedure.

A variety of methods have been tried in an effort to ease the patient's discomfort. One method is to allow the patient to control some facet of the breast compressive forces. Another method is to train the technician to be more empathetic to each particular patient's demeanor. Still another method is to provide a breast cushion interface plate as disclosed in U.S. Pat. Nos. 5,185,776 and 5,377,254 and 6,577,702. Also a unique type of machine for improving the mammography procedure was disclosed in U.S. Pat. No. 5,590,166 wherein the bucky (the support and container for the X-ray film or digital imaging device) and the breast compression paddle, can move toward each other “simultaneously at a substantially equal but oppositely directed speed” for compressing the breast.

The present invention is directed to the same important purpose as prior art methods and apparatus; that is, making the mammography procedure less painful. The inventive method requires minimal additional accessories to the mammography machine equipment, and requires minimal additional training or experience in utilizing the method. Further, the quality of the images obtained by the digital detectors or film of the X-ray machine are improved since, with the new compression technique, the mass of the breast will tend to be spread out somewhat more uniformly than in prior art procedures. Most importantly, the unique breast interface and compression interface plate disclosed herein will be more comfortable for the patient.

### SUMMARY OF THE INVENTION

A method and apparatus are disclosed wherein a movable breast interface plate mounted on the bucky is selectively actuated and moved toward the associated compression paddle during the breast compression procedure. This provides cooperating, more uniform, more evenly distributed, and more comfortable compressive and shear forces to the breast.

The foregoing features and advantages of the present invention will be apparent from the following more particular description of the invention. The accompanying drawings, listed herein below, are useful in explaining the invention.

### DRAWINGS

FIG. 1 is a sketch showing the inventive interface assembly mounted on a standard mammography machine;

FIG. 2 is a sketch, labeled prior art, showing a mammography machine as disclosed in U.S. Pat. No. 5,590,166 wherein the bucky is movable on the C-arm;

FIG. 3, labeled prior art, is a sketch indicating a compression paddle compressing a patient's breast a desired amount;

FIG. 4 is a sketch showing the inventive apparatus and concept of a movable breast interface plate mounted on the bucky;

FIG. 5 is a sketch showing the position of the plate on the bucky lifting the breast toward the compression paddle in accordance with the inventive method;

FIG. 6 shows a sketch of a breast useful in explaining the method of the invention.

FIG. 7 is a sketch depicting, by the arrow line, the compression and shear forces on a patient's breast as in prior methods;

FIG. 8 depicts the complementary and cooperating compression forces developed by moving (elevating) the plate on the bucky, in accordance with the inventive method;

FIG. 9 depicts one early embodiment of a breast interface plate

FIG. 10 shows a view of the interface plate of FIG. 9 in an expanded mode;

FIG. 11 shows a view of the interface plate of FIG. 9 mounted on the bucky and in an expanded mode;

FIG. 12 shows a view of FIG. 10 in partial cross section to more clearly show an expanded configuration of the interface plate;

FIG. 13 shows an embodiment of the interface plate wherein a portion of the top surface is contoured to cradle a patient's breast;

FIG. 14 shows an embodiment of the breast interface assembly wherein linear actuators are positioned to elevate or raise a breast interface plate comprising a plate;

FIG. 15 shows a relative enlarged view of the mounting of a linear actuator on the bucky;

FIG. 16 is a front view of the interface plate comprising a plate of FIG. 14 when the plate is in a first lowered mode contiguous to the bucky;

FIG. 17 is a front view of the interface plate of FIG. 14 in an elevated mode raised upwardly from the bucky; and

FIG. 18 shows a linear actuator mounting, wherein a narrow strip is provided to support the linear actuator adjacent to the bucky.

### DESCRIPTION OF THE INVENTION

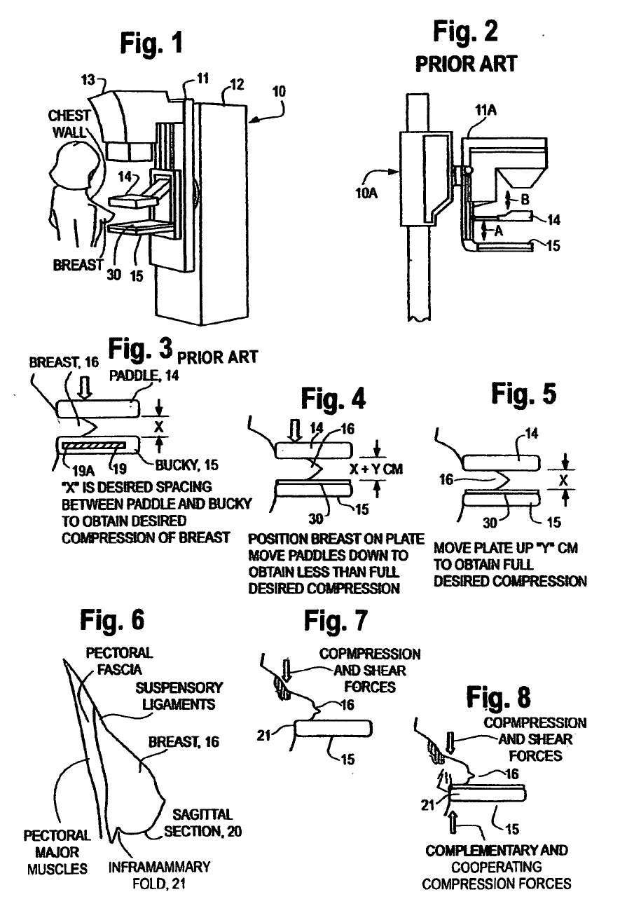

FIG. 1 depicts the inventive relatively rigid plastic interface plate 30 mounted on a standard mammography machine 10. The machine 10 includes a C-arm 11 mounted on a base 12. An X-ray source 13 mounted on the upper end of the C-arm 11 selectively provides an X-ray beam toward a bucky 15 that is supported at the lower end of the C-arm. The bucky is fixed or stationary relative to the C-arm and the machine provides a fixed X-ray source to image detector distance.

The C-arm 11, and the affixed bucky 15, are rotatable and tiltable for, e.g., taking oblique images of the breast. Contained within the bucky 15 is a suitable known type of image detecting and recording device 19 (such as a digital image detector or a film cassette) that may be inserted through a slot 19A into the bucky, see FIG. 3.

A patient is depicted by the dotted lines of FIG. 1. The bucky 15 is oriented to provide a support surface or table for the patient's breast during the mammography procedure.

The bucky 15 is stationary or fixed relative to the C-arm, a breast compression paddle 14, mounted on the center or bight of the C-arm 11, is movable relatively toward and away from the bucky 15. The compression paddle 14 includes a bottom surface that is operated to press on and compress the patient's breast. In the prior art the paddle 14 compresses the breast between it and the bucky 15, as depicted in FIG. 3. In the inventive system, the paddle 14 cooperates with an interface plate generally labeled 30 in FIG. 1, to compress the breast there between, as will be further explained.

The present invention discloses a method and apparatus for compressing the breast during the mammography procedure wherein a breast interface plate mounted on the bucky is controllably movable relative to the bucky.

In the prior art, a first step in the mammography procedure for taking a craniocaudal (head-to-toe orientation) image is to position the patient's breast on the bucky 15 such that the weight (mass) of the breast is supported on the bucky. As depicted in FIG. 3, the next step is to firm the compression paddle 14 against the chest wall, the upper surface of the breast and the suspensory ligaments of the breast. Next, the compression paddle 14 is powered to move down substantially parallel to and adjacent to the chest wall, engaging the upper posterior portion of the breast, hence pressing on and forcing the breast downward.

As noted in FIG. 3, in order to provide a desired spacing, indicated as “X”, between the compression paddle 14 and bucky 15 the breast is compressed to the desired position for taking an X-ray image. This develops compression forces and shear forces on the chest wall and the breast. The human skin does not stretch easily, and depending on the size of the breast and the condition of the muscles and ligaments, the shear forces applied can be substantial as the compression paddle 14 is forced down to compress the breast to a desired position. Many patients complain that this method is quite painful.

In the inventive method, and as depicted in FIG. 4, the breast is positioned on a breast interface plate 30 positioned on the bucky 15, and the compression paddle 14 is moved to compress the breast. Importantly however, in the inventive method, before the full desired selected compression of the breast is attained, the movement of the compression paddle 14 is stopped at some intermediate position where the compression force has compressed the breast to less than the full desired compression. A pausing of the procedure at this point also allows the technician to check whether the breast is compressing properly. Next, and as depicted in FIG. 5, breast interface plate 30 mounted on the bucky 15 is elevated or moved toward the paddle 14 to obtain the full desired compression. The interface plate 30 combines or cooperates with the compression paddle 15 to provide the selected compression.

FIGS. 4 and 5, graphically show the inventive method. A majority, but less than the full desired compression, is indicated in FIG. 4 as “X+Y” cm. It should be understood that because of the different sizes, configurations and firmness of patients' breasts both “X” and “Y” are variables. In use, the technician determines the required compression for each individual breast. At the intermediate position, it is approximated that 70% to 85% of the desired compression has been attained; these indicated percentages of compression are not preset, but rather the technician determines the amount of compression through training and experience based on the required compression for proper imaging, while also taking into consideration the patient's comfort.

Patients usually appear to sense a higher degree of pain at the higher compressive forces when the breast is being compressed to the full compression. Patients appear to sense much lesser pain or discomfort at an intermediate position of the sequence. However, at the intermediate position of the paddle 14 (as indicated in FIG. 4) the breast has not been sufficiently compressed for taking the X-ray image. Accordingly, in the next step of the inventive procedure, movement of the paddle 14 is stopped at the aforementioned intermediate position, and the paddle now becomes a fixed upper support against which the breast is compressed by the interface plate 30. In the following step, depicted in FIG. 5, the breast interface plate 30 on the bucky 15 is caused to move upwardly from its initial position (indicated by the dotted lines) to the “full” position indicated by the solid lines, to compress the breast there between. The breast interface plate 30 on the bucky 15 is moved toward the compression paddle 14 a distance of “Y” cm, an amount equal to the amount necessary to compress the breast to a position to provide the desired separation “X”. The breast is now in position for taking an X-ray image. After the X-ray procedure is completed, the compression paddle and the interface plate are returned to their initial positions.

For additional description, refer to FIG. 6 that shows an outline of a patient's breast 16. Essentially, in the latter steps of the inventive method, the initial roles of the compression paddle 14 and the bucky 15 are effectively reversed. That is, the compression paddle 14 becomes a support reference surface and the breast interface plate 30 on the bucky 15 is moved to apply the additional compressive force to the sagittal section 20 (the underside) of the breast.

Refer now also to FIGS. 7 and 8. The inventive method tends to decrease the compressive and shear forces applied to the upper section of the breast including the major pectoral muscles and suspensory ligaments of the breast, and in so doing reduces the pain felt by the patient. Note that as depicted in FIG. 6, the sagittal section 20 of the breast is normally lower than the inframammary fold 21 that joins the sagittal section to the chest wall. As depicted in FIG. 7, when the breast is positioned on the breast interface plate 30, the inframammary fold 21 and the sagittal section 20 are essentially level with the upper surface of the bucky.

In the prior methods, when the compression paddle 14 is lowered toward the bucky 15, essentially all the compressive and shear forces are applied to the top of the breast; that is, the forces are effective on the suspensory ligaments, tissue and muscles of the breast, see FIG. 7. There are limited compressive and shear forces on the sagittal section 20 of the breast and on the tissue and ligaments adjacent the inframammary fold 21.

The inventive method provides a procedure for distributing the compressive and shear forces applied to the breast. More specifically, in the inventive method after the compression paddle 14 is stopped at an intermediate position, the breast interface plate 30 on the bucky 15 is caused to move up to provide an active compression force to the sagittal section 20 of the breast. This is indicated in FIG. 8 by the arrow line labeled “complementary and cooperating compression forces”. Upward movement of the breast interface plate compensates for the distance that the compression paddle 14 would have moved in the prior methods.

As depicted in FIG. 8, compression and shear forces will still be applied to the top muscles and ligaments of the breast which will still tend to be stretched, but more moderately. Note that as the breast interface plate 30 on the bucky 15 is caused to move up, the tissue and ligaments on the sagittal section 20 and the adjacent chest wall will also be subjected to shearing and compressive forces. However, the level of these shearing and compressive forces will be more distributed and balanced. Likewise, the forces applied to the upper posterior of the breast will be substantially less than the forces applied by the prior art. As stated above, since the size and types of patients' breast vary so considerably, the actual distance of movement of the breast interface plate 30 on the bucky 15 varies.

As further explanation, the breast may be considered as a non-symmetrical object effectively suspended from a vertical surface. Muscle, ligaments and tissue that extend from the chest wall above the breast principally suspend the breast, see FIG. 6. In most prior mammography compression procedures, the sagittal section of the breast has been “passively” supported on the bucky, and all the compressive forces have been applied to the upper posterior section of the breast, see FIG. 7. In the prior art, as the compression paddle is moved to compress the breast, the edge of the paddle engages the upper posterior section of the breast at an angle, and the compressive and shear forces that are developed by the paddle will tend to stretch the muscle, ligaments and tissue of the upper posterior of the breast while minimal forces effect the sagittal section of the breast. As the compression paddle is moved, the shear forces tend to cause stretching of the muscles, ligaments and tissue suspending the breast, which is a major source of the pain experienced by the patient. During this same period of paddle movement, other portions of the breast including the sagittal section may only be minimally stressed.

In contrast to the prior art, in the present invention a basic principle is to more evenly distribute the forces applied to the breast during the compression procedure. This reduces the shear forces applied to the breast suspending muscles, ligaments and tissue and applies additional compressive forces to other parts of the breast. The total effective compression force on the breast remains essentially the same, but it is more uniformly distributed.

In almost all mammography machines the bucky 15 is fixedly mounted on the C-arm, that is, it is in fixed or stationary position on the C-arm, and only the paddle 14 is movable toward and away from bucky 15. There is however, one known X-ray machine that comprises a bucky that is movable on the C-arm relative to the source of X-rays. Refer now to FIG. 2 that shows the known machine as disclosed in U.S. Pat. No. 5,590,166 made by Instrumentarium Imaging Inc., now GE Medical Systems Inc. A significant distinction between the machine 10 of FIG. 1 and machine 10A of FIG. 2 is that in machine 10 the bucky is stationary or fixed on the C-arm 11. In contrast, in machine 10A, the bucky is movable on the C-arm 11A. As indicated by the arrow labeled “A” in FIG. 2, the bucky 15 of machine 10A is movable toward and away from the compression paddle 14. Also, as indicated by the arrow labeled “B” the compression paddle 15 is movable toward and away from bucky 15. U.S. Pat. No. 5,590,166 states that machine 10A includes a linear motorized drive to move the compression paddle 14 and the bucky 15 simultaneously in opposite directions at a substantially equal speed for compressing the breast between the compression paddle and the bucky. Machine 10A suggests an action such as that of closing a pair of pliers on a symmetrical object to provide the compression. In contrast to the method of U.S. Pat. No. 5,590,166, the present invention controllably moves the compression paddle and breast interface different distances at different speeds, and importantly, the compression paddle will still exert the major active compressive force. This compensates for the different structure and sensitivities of the various sections of the breast to thereby reduce pain and discomfort during the compression procedure. In an article Diamond Breast Care of Instrumentarium Imaging Co. titled “more breast tissue with improved comfort” a variation of the method described in U.S. Pat. No. 5,590,166 (assigned to Instrumentarium Imaging Co.) is disclosed wherein the breast is placed on a bucky to open the infra-mammary fold, then the breast is compressed slightly with the compression paddle to hold the breast in place, then the final compression is applied by the bucky from below. As is understood, the method taught in said article has found limited use. A reason may be that the ligament structure of the breast is not amenable to major lifting and compression from below since the area of the infra-mammary fold may then be subject to tearing of the tissue. The method disclosed in the foregoing article is different from the method of the invention. In the inventive method the major active compressive force is applied by the compression paddle, and importantly the interface plate is only moved a relatively minor amount after the major compressive force of the compression paddle is exerted.

The inventive method utilizes a movable breast interface plate that is mountable on a bucky. The apparatus also discloses drive means for controllably moving the interface plate away and toward the top surface of the bucky. More specifically, the present invention discloses a breast interface apparatus including a radiolucent (X-ray transparent) plate 32A (se FIGS. 14-17) that is mountable on the bucky 15. The interface plates 30 of FIG. 1 and 32A of FIGS. 14-17 are similar and function as a controllably movable breast interface for compressing a patient's breast. Power means are provided for controllably moving the interface plate 14A away and towards the bucky from the compression paddle to practice the inventive method.

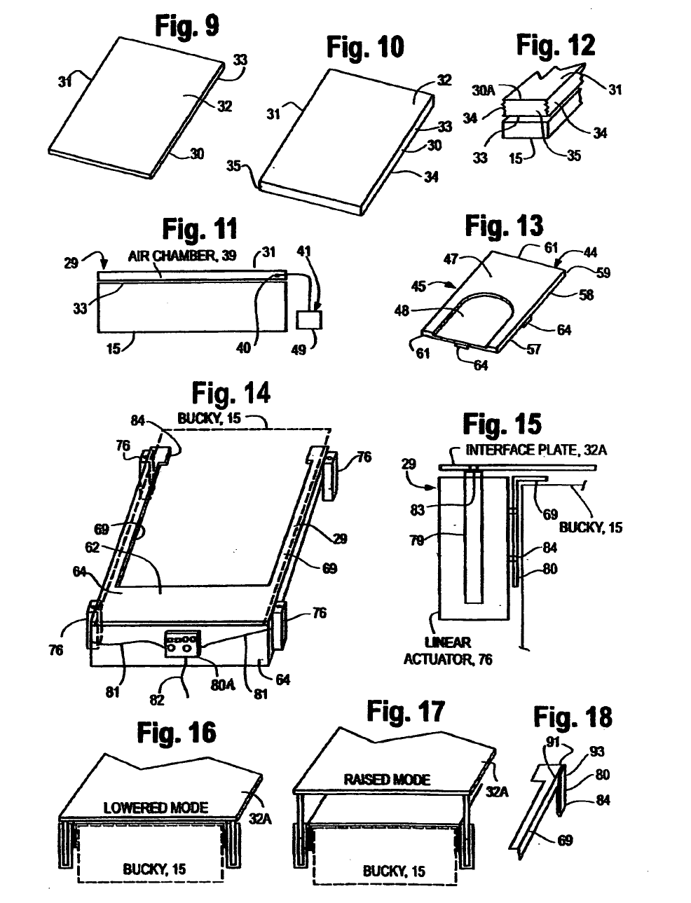

The inventive apparatus will now be further described. Refer to FIGS. 9-18 that show an interface assembly 29 that includes the selectively movable breast interface plate.

Referring to FIGS. 9-13, in initial experimental embodiments an expandable canister 31 was provided to serve as an interface plate 30. The canister 31 is shown in a non-expanded mode in FIG. 9 and in an expanded mode in FIGS. 10 and 11. Canister 31 includes a top surface 32 a bottom surface 33, sides 34 and ends 35. FIG. 11 shows that the canister form an air chamber 39 that is supplied by an air line controlled by a suitable compressor 49. FIG. 12 shows sides 34 which are pleated to enhance flexibility.

FIG. 13 shows a canister 44 that is a modification of the canister 31 shown in FIG. 9-12. Canister 44 includes a top plate 45 formed of two sections 47 and 48. One section 48 is made in a partially rounded pattern to accommodate the flattened form of a breast, and is made of flexible material. The other section 47, including the part surrounding the first section 48 is made of relatively rigid material. Sections 47 and 48 are bonded together. The front end 61 of canister 44 is made of a flexible material and bonded to flexible sides 58, and to the top and bottom plates. The bottom plate 57, sides and end 59 are of a rigid plastic. End 61 is of a flexible material similar to section 48. When canister 44 is in a non-expanded mode, as shown in FIG. 13, section 48 forms a cradle for receiving the patient's breast. When canister 44 is inflated, section 48 is the surface that is inflated to form a firm lifting plate for the patient's breast. The embodiment of FIG. 13 was found to be difficult to make.

Tabs 64 were affixed to the sides and ends of canister 44 to extend down alongside the bucky to hold the canister in position on the bucky.

It was found that although the structures of FIGS. 9-13 are feasible, the operation becomes complex. More specifically, in the embodiments of FIGS. 9-13 it has been found difficult to raise the interface plate in a mode that maintains the plate parallel to the top surface of the bucky 15. The interface plate 30 tends to expand or raise at an angle, or slants, rather than parallel to the top surface of the bucky.

A preferred embodiment of the movable breast interface plate is shown in FIGS. 14-18. This latter embodiment of interface assembly 29 comprises the interface plate labeled 32A, that is similar to plate 30, and is also of relatively rigid plastic. Plate 32A is also radiolucent (X-ray transparent), and is controllably movable by miniature linear actuators 76 such as electric linear actuators or pneumatic air cylinders. Plate 32A is indicated as affixed to actuators 76, as by gluing or by a suitable screw.

Linear actuators of many types are well known in the art and made by many manufacturers. Manufacturers such as the Bimba Manufacturing Co., Monee, Ill. and Parker-Hannifin Corp., Actuator Division, Wadsworth, Ohio make pneumatic air cylinders. Parker-Hannifin Corp. also offers a full line of electric linear actuators as well as miniature pneumatic cylinders. Electric linearly actuators and air cylinders are commonly used in many precise positioning and actuating applications and devices. One common type of electric linear actuator provides linear motion via a motor driven screw assembly.

Refer now to FIG. 14; the interface assembly 29 comprises a generally U-shaped frame 62 that can be mounted on a bucky 15. The frame 62 has a bight or end 64 that is rectangularly dimensioned to fit over the end of the bucky 15. The top of bight 64 is supportable on the bucky, and the sides extend alongside the sides of the bucky. The frame 62 includes two arms 69 that are of inverted “L-shape” cross section and that extend along the edge of the bucky. The top of the arms 69 is supportable on the top of the bucky and the sides of the arms 69 abut the sides of the bucky. The bight 64 and the arms 69 are dimension so as not to impede the use of slots on the side of the bucky 15 that receive the film cassette or digital imager.

Linear actuators 76 are affixed to the frame 62, two at the ends of arms 69, and two at sides of the bight 64. The actuators may be affixed on support strips 80 that extend downwardly from arms 69, see also FIGS. 15 and 18. In the embodiment of the assembly 29, actuators 76 shown in FIGS. 14 and 15 are electric linear actuators and are powered through suitable leads 81 from a control box 80A. Control box 80A is either connected to an electric outlet via lead 82, or is battery powered. FIGS. 15 and 18 also indicate the attachment (by suitable pins or screws 84) of a linear actuator 76 to the strip/flange 80 of arm 69, and the mounting of the arms 69 on the bucky 15. Further, FIG. 15 depicts the coupling or connection 83 of the movable rod 79 of actuator 76 to the interface plate 32A. The connection 83 can be a screw, a pin inserted into a slot in interface plate 32A, or by gluing. The linear actuators 76 controllably raise and set or lower the interface plate 32A, for the purposes described above.

The movable interface plate 32A that is moved utilizing small components is necessary in view of the limited space available for positioning and operating the interface plate, and because there should be minimal attenuation or interference with the X-ray beam.

As mentioned above, the actuators 76 can also be miniature air cylinders powered by compressed-air pumps or devices that are commercially available from various sources, as mentioned above. The air cylinders are positioned similarly as the electric actuators described above and are actuated by a suitable source of air pressure 49, as depicted in FIG. 11, to raise and lower the interface plate 32A.

FIG. 16 shows the interface plate 32A in a position adjacent the bucky 15, and FIG. 17 shows the interface plate 32A in a raised or elevated position relative to the bucky 15 to provide compressive forces to the breast. Briefly stated, the linear actuators 76 are energized to raise the interface plate 32 in an essentially parallel relation to the top surface of the bucky 15 to a set position as determined by the operator technician. The technician then stops the actuators 76 to thereby set and lock the plate 32A in a selected position. The apparatus is ready for taking an X-ray image.

FIG. 18 shows a support strip 80 formed at the ends of the arms 69. A linear actuator 76 is affixed to each strip 80 by pins or screws 84, as depicted in FIG. 15. Strips 80 are relatively narrow and are formed at the ends of arms 69 to extend downwardly alongside the bucky 15. The positioning of the actuators 76 on the ends of arms 69 does not impede the placing or removing film cassettes or digital imaging accessories through the bucky slot 19A, see FIG. 3. A hinge indicated at 93, in FIG. 18 may be installed and the strip 80 cut on one side and at the top of adjacent arm 69, as at 91, to permit the strip 80 and the affixed linear actuator to swing away from the side of the bucky when inserting or removing film etc.

In FIGS. 14-18 the interface assembly 29 comprises a single radiolucent (X-ray transparent) interface plate 32A that is mounted in the path of the X-ray beam. A single plate 32A tends to minimize the attenuation of the X-ray beam.

The operation or use of the apparatus of FIGS. 14-18 will now be described. Refer to FIG. 14 as well as FIG. 1 interface assembly 29 is first mounted on the bucky 15. The patient's breast is then positioned on the breast interface plate 32. Next the compression paddle 14 is lowered to engage and compress the patient's breast. As stated above, the compression paddle 14 is moved downwardly toward the interface plate 32A to obtain the majority of, but less than the full desired compression. Next, as also described above, movement of the compression paddle 14 is paused. The linear actuators 81 are then energized to activate the associated drive rods 83 drive the interface plate 32A to a selected spaced distance from the bucky 15. The interface plate 32A is moved up or raised in a consistent parallel orientation to the top surface of the bucky 15, as is desired for taking proper X-ray mammography images. In one embodiment, plate 32A is raised upwardly about 1-4 cm, that is to a position spaced from the bucky about 1-4 cm to push upwardly against the bottom of the breast. The selected the position is dependent on the shape and size of the breast. Note, that the compression paddle 14 moves a relatively much larger distance than the interface plate 32A, and the interface plate 32A effectively adds the final percentage of the total compressive force.

The actuators 76 are then stopped to set and lock the interface plate 32A at the selected position determined by the technician. The breast is now in the proper position for X-ray imaging. After the imaging procedure is completed, the compression paddle 14 and the interface plate 32A are returned to their respective initial positions.

The position of the interface plate 32A can be accurately adjusted by forward and reverse tenderization of the linear actuators, as is known. The exact amount of movement of the interface plate 32A to obtain the required compression of a patient's breast is determined by training and experience. As stated above, the precise or particular relative movement of the paddle 14 and the interface plate 32A varies dependent on the size and configuration of the breast.

As a modification of the present inventive method, the compression paddle 14 and the interface plate 32A can be caused to move concurrently, that is, the interface plate could be rising and moving at the same time that the bucky is moving down to compress the breast. However, the amount of movement and the speed of movement of the two components would be different. Moreover, because of the variability of the size, configuration and firmness of the patients' breasts (and the patient's different comfort levels and pain thresholds), concurrent movement appears to be a more critical method.

While the invention has been particularly shown and described with reference to preferred embodiments thereof, it will be understood by those skilled in the art that various changes in form and details may be made therein without departing from the spirit and scope of the invention.

1. In an X-ray mammography machine for taking an image of a patient's breast, said machine having a C-arm, an X-ray source mounted on one free end of said C-arm for providing a beam of X-rays for imaging a patients breast, a bucky mounted on the other free end of said C-arm, said bucky housing a digital imaging detector or a film cassette for sensing X-ray images, said X-ray source and said bucky being fixed relative to one another a fixed distance apart that provides a fixed X-ray source to image detector distance, said bucky having a top mounting surface; and, a movable compression paddle mounted on said C-arm, an apparatus for facilitating compression of the patient's breast comprising,
a) a radiolucent rigid plate positionable on said bucky,
b) said plate being movable in a parallel relation to said top mounting surface of said stationary bucky to selectively raise relative to said top mounting surface;
c) said plate providing an interface for supporting and carrying the breast upwardly relative to the top mounting surface of said stationary bucky;
d) linear actuators mountable on said bucky and outside the path of said X-ray beam, said actuators connected to said plate for raising and lowering said plate in parallel orientation relative to said top mounting surface of said bucky,
e) said actuators operable to raise said plate to a selected set position parallel to the top mounting surface of said bucky and spaced relative to said bucky, and to retain said plate in said set position,
f) said plate and said actuators providing an active moving compressive force to the lower side of the patient's breast and parallel to the top mounting surface of said bucky in preparation for taking an X-ray image as said plate is being moved to said set position, said set position being in essentially parallel spaced relation to said top mounting surface of said bucky; and
g) means for controllably energizing said actuators for selectively raising and setting or lowering said interface plate relative to said bucky to provide, in cooperation with said compression paddle, a selected compression of a patient's breast.

2. Apparatus as in claim 1 wherein said plate is operatively movable in the range of 1-4 cm above the top surface of said bucky and spaced from said bucky.

3. Apparatus as in claim 1 wherein said plate comprises a relatively flat plate free of downward side extensions.

4. Apparatus as in claim 1 wherein said linear actuators comprise electric linear actuators.

5. Apparatus as in claim 1 wherein said linear actuator means comprise pneumatic air cylinders.

6. A breast interface assembly for mounting on the bucky of a mammography machine, said machine having a C-arm member, the upper arm of said member supporting an X-ray source providing a beam of X-rays, the lower arm of said member supporting bucky that is fixed to said lower arm, a compression paddle mountable on the bight portion of the C-arm to be movable thereon, said bucky having an top mounting surface, and said compression paddle being movable downwardly toward said bucky to compress a patient's breast, said interface assembly comprising,
a) a breast interface plate positionable on said top mounting surface of said bucky for supporting a patient's breast;
b) linear actuator means mountable on said bucky, said linear actuator means connected to controllably and selectively drive said interface plate in a parallel relation away and towards the top surface of said bucky to provide a selected spacing between said bucky and said interface plate;
d) a framework for supporting said linear actuator means on the surface of said bucky, said framework being mountable outside the path of said X-ray beam; and
e) said actuators means controllably raising and setting said interface plate in a selected breast compressing operating position spaced from the top surface of said bucky.

7. In an X-ray mammography machine for taking an image of a patient's breast, said machine having a C-arm, an X-ray source mounted on one free end of said C-arm for providing a beam of X-rays for imaging a patients breast, a bucky mounted in a fixed position on the other free end of said C-arm, said X-ray source and said bucky being a fixed distance apart, said bucky having a top mounting surface thereon, and a movable compression paddle mounted on said C-arm, said C-arm being movable from a first position to a second or breast compressing position, a method of compressing a patient's breast to a full selected compression for taking an X-ray image, said method comprising
a) mounting in an initial position a selectively movable and relatively rigid breast interface plate on said fixed bucky;
b) positioning a patient's breast on said movable interface plate;
c) retaining said X-ray source and said bucky in a fixed position relative to one another;
d) moving said compression paddle toward said interface plate from a first position toward a selected position to compress the breast there between;
e) stopping movement of said compression paddle after the major part of the full selected compression is attained, and after said compression paddle has stopped;
e) raising said interface plate from its said initial position toward said stopped compression paddle to provide a selected spacing between the top surface of said bucky and said interface plate to raise the underside of the patient's breast upwardly to obtain said full compression of the breast;
f) setting said interface plate at said selected position for taking an X-ray image; and
g) returning said compression paddle and said interface plate to their initial positions to release the compressive force.

8. A method as in claim 7 further including the step of
a) checking said compressive force after said compression paddle is stopped when he compression is less than said full selected compression; and
b) moving said interface plate upwardly toward said compression paddle to provide the final additional percentage of compressive force.

9. A method as in claim 7 wherein said interface plate has a non-elevated mode and an elevated mode, and the method including the steps of
a) mounting said interface plate on said bucky when said interface plate is in said first non-elevated mode; and,
b) moving said interface plate to its elevated mode upwardly against the patient's breast and toward said compression paddle.

10. A method as in claim 7 wherein said interface plate and said compression paddle are moved at different speeds and for different distances.

11. A method as in claim 7 including the step of
a) raising the interface plate toward said compression paddle in the range of 1 to 4 cm.

12. A method as in claim 7 further including the step of
a) raising said interface plate to be consistently parallel to said top surface of said bucky.
